(19) United States
(12) Patent Application Publication
(10) Pub. No.: US 2026/0198899 A1
(43) Pub. Date: Jul. 16, 2026

(54) LONGITUDINAL QUANTITATIVE ULTRASOUND MONITORING

(71) Applicant: Siemens Medical Solutions USA, Inc., Malvern, PA (US)

(72) Inventors: Yassin Labyed, Carlsbad, CA (US); Ravi Kumar Shamanna, Covington, WA (US); Gilles D. Guenette, Fall City, WA (US)

(21) Appl. No.: 19/024,208

(22) Filed: Jan. 16, 2025

(51) Int. Cl. A61B 8/00; A61B 8/08; G16H 50/20

(52) U.S. Cl. CPC A61B 8/5246; A61B 8/08; A61B 8/463; A61B 8/469; A61B 8/54; A61B 8/565; G16H 50/20

(57) ABSTRACT

For longitudinal monitoring in quantitative ultrasound (QUS) imaging. User variability is reduced by loading the same settings from a baseline or earlier examination for a follow-up examination. Artificial intelligence (AI), given these same settings, guides placement of the field of view (FOV) and/or region of interest (ROI) in the patient for the follow-up examination. User variability is reduced, allowing for more accurate comparison between different examinations. Tools or applications using the longitudinal study may be more accurate, so the tools or applications are made more accessible on a variety of platforms rather than just the ultrasound scanner.

### BACKGROUND

The present document relates to quantitative ultrasound (QUS) imaging. In QUS imaging, the detected information is further processed to quantify a biomarker or characteristic of the tissue being imaged. Rather than merely providing a B-mode image of the tissue, a characteristic of that tissue is imaged. For example, shear wave speed in the tissue is calculated using ultrasound imaging. Other examples include strain, attenuation, backscatter, or ultrasound-derived fat fraction.

For quantitative ultrasound imaging, a user typically positions a region of interest (ROI) in a B-mode image. To avoid delays or processing complications for quantification over the entire field of view (FOV) of the B-mode image, the user-positioned ROI defines the region of tissue for quantification.

QUS biomarkers hold promise not only for screening and diagnosis, but also for monitoring disease progression or response to treatments from lifestyle, dietary, and/or pharmaceutical interventions. Since different QUS examinations are performed before and after treatment, the monitoring is sensitive to proper placement of the FOV and ROI in the different examinations. The matching of ROIs over time or in different examinations may be subjective and inaccurate. As a result, less diagnostically or prognostically reliable comparison of QUS biomarkers is available. While visualization tools provide measurements from different examinations, the measurements may not be directly comparable due to user variability.

### SUMMARY

By way of introduction, the preferred embodiments described below include methods, computer readable storage media, instructions, and systems for longitudinal monitoring in QUS imaging. User variability is reduced by loading the same settings from a baseline or earlier examination for a follow-up examination. Artificial intelligence (AI), given these same settings, guides placement of the FOV and/or ROI in the patient for the follow-up examination. User variability is reduced, allowing for more accurate comparison between different examinations. Tools or applications using the longitudinal study may be more accurate, so the tools or applications are made more accessible on a variety of platforms rather than just the ultrasound scanner.

In a first aspect, a method is provided for longitudinal monitoring in quantitative ultrasound imaging with an ultrasound scanner. First values for settings used in a previous quantitative ultrasound examination of a patient are acquired. The ultrasound scanner is configured for a current quantitative ultrasound examination of the patient with the first values for the settings. A machine-learned model guides placement of a region of interest relative to the patient. The ultrasound scanner as configured by the first values and using the region of interest as placed by the guidance is used to perform the current quantitative ultrasound examination of the patient. A comparison of a first measure from the current quantitative ultrasound examination with a second measure from the previous quantitative ultrasound examination is displayed.

In a second aspect, a method is provided for longitudinal monitoring in quantitative ultrasound imaging with an ultrasound scanner. Settings for the ultrasound scanner are reloaded from a previous scan session. An artificial intelligence guides placement of a current position of a region of interest relative to a patient based on data from the previous scan session. Quantitative analysis from the quantitative ultrasound imaging by the ultrasound scanner using the reloaded settings with the current position of the region of interest is displayed.

In a third aspect, a system is provided for longitudinal monitoring in quantitative ultrasound imaging. An ultrasound imaging system is configured to reload settings from a baseline quantitative ultrasound imaging session for a follow-up quantitative ultrasound imaging session. An image processor is configured by a machine-learned model to identify an anatomical structure and guide a user to a view of the patient and position of a region of interest in the view. The view and position for the follow-up quantitative ultrasound imaging session is guided to match the baseline quantitative ultrasound imaging session. A display is configured to display a change in a quantitative ultrasound measurement from the baseline quantitative ultrasound imaging session to the follow-up quantitative ultrasound imaging session.

Any one or more of the aspects or concepts summarized above or in the Illustrative Embodiments below may be used alone or in combination. The aspects or concepts described for one Illustrative Embodiment or aspect may be used in other embodiments or aspects. The aspects or concepts described for a method or system may be used in others of a system, method, computer program, or non-transitory computer readable storage medium.

The present invention is defined by the following claims, and nothing in this section should be taken as limitations on those claims. Further aspects and advantages of the invention are disclosed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one implementation of a method for longitudinal monitoring in QUS imaging with an ultrasound scanner;

FIG. 2 is a flow chart diagram of another implementation of a method for longitudinal monitoring in QUS imaging with an ultrasound scanner;

FIG. 3 illustrates an example tool or application reporting a measurement in QUS; and

FIG. 4 is a block diagram of one implementation of a system for longitudinal monitoring in QUS imaging.

### DETAILED DESCRIPTION OF THE DRAWINGS AND PRESENTLY PREFERRED EMBODIMENTS

In QUS imaging, monitoring assists with various aspects to reduce human variability. Saving and reloading scanner settings for QUS examination and positioning the ROI based on predefined criteria may ensure consistent imaging features across follow-up studies in longitudinal QUS. For example, scanner settings of QUS are automatically saved as part of the patient's study. These scanner settings from previous baseline or follow-up exams are reloaded for a current exam. Artificial intelligence (AI) positions the QUS ROI based on predefined criteria, such as the same criteria used in a previous (e.g., baseline) exam. The features of the imaging views used in QUS acquisition as part of the patient's study, such as features derived or detected by the AI, are saved for repeatable positioning of the FOV and/or ROI. The user is guided to the optimal view (e.g., FOV) and QUS ROI placement to maximize feature similarity between exams.

In one implementation, system software, AI guidance, and quantification and visualization software are combined. The settings from a previous scan session are reloaded. AI guides the user to position the ROI based on data from a previous session. Quantitative analysis and visualization software is provided on the ultrasound imaging system, mobile application, or a web application for viewing the resulting longitudinal QUS measurements. The measurements from different times more accurately reflect change in tissue due to the use of the same settings and measurement at the same tissue (i.e., same ROI).

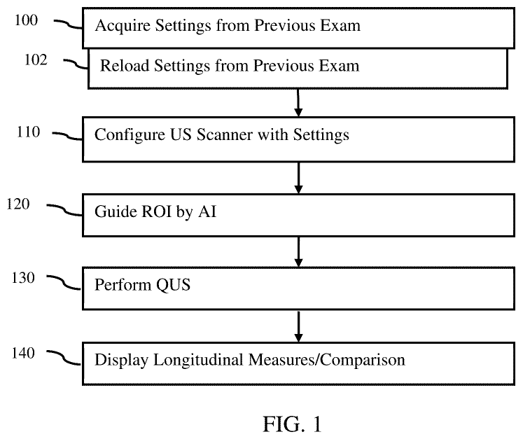

FIG. 1 shows one implementation of a method for longitudinal monitoring in QUS imaging with an ultrasound scanner. For longitudinal study, the same anatomy is imaged with QUS imaging at different times or for different examinations. The examinations may be separated by a treatment and/or hour or more. The examinations may be performed by the same or different sonographers using the same or different ultrasound scanner. To reduce human variability, the settings from a previous scan are acquired and used for the current (subsequent) scan. AI assists in FOV and/or ROI placement so that the measurements are for the same tissue. The display of comparison of the same measure at the different times may assist in diagnosis, prognosis, and/or treatment.

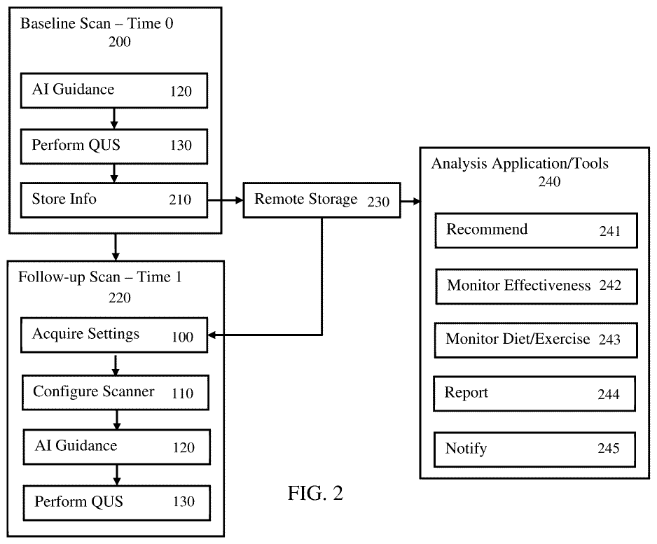

FIG. 1 is directed to the current or subsequent examination. FIG. 2 shows an implementation where both the baseline (e.g., initial or previous but not initial) and subsequent (e.g., current or follow-up) examinations are performed.

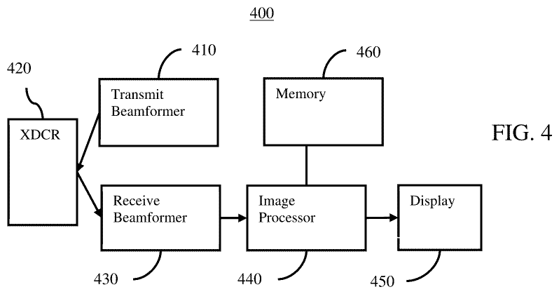

The methods of FIGS. 1 and/or 2 are performed by the system shown in FIG. 4 or a different system. For example, a medical diagnostic ultrasound imaging system performs the acts of FIG. 1 and corresponding acts in FIG. 2. The same or different medical diagnostic ultrasound imaging system performs the baseline acts of FIG. 2. The medical diagnostic ultrasound imaging system or another device may perform the analysis. For example, a mobile phone, computer (e.g., desktop, tablet, or workstation), or server may perform the analysis using an application (e.g., program) or tool 240. Other devices may perform any of the acts, such as a picture archiving and communications system (PACS) or computerized medical records database providing the remote storage 230.

The acts are performed in the order shown (numerical order) or another order. For example, acts 241-245 are performed simultaneously or in any order.

Additional, different, or fewer acts may be used. For example, the acts of FIG. 1 are performed without or based on previous performance on the baseline scan acts of FIG. 2. As another example, the use of reloading settings is provided without AI guidance, or vise versa. In yet another example, any or none of the acts for analysis are provided.

In act 200, the ultrasound scanner performs a baseline or initial QUS scan of the patient. The user controls and/or configures the ultrasound scanner for scanning. Presets or default settings may be used. The user may alter any of the settings. After locating the FOV to scan the desired tissue (e.g., liver segment), the ROI is placed, and the QUS measurement is performed.

To locate the ROI for quantitative imaging, ultrasound data representing or responsive to a patient is acquired. This scan is an initial scan, such as a first scan or a later scan once quantitative imaging is to be used. For example, the scanning is repeated as a sonographer positions the transducer to scan the desired region of the patient. The FOV for the scanning is positioned over the organ or organs of interest. Once the object of interest is in the FOV, the ultrasound data to be used for locating the ROI is available from the scanning or is acquired by further scanning.

The scan for ultrasound data to locate the ROI is of the entire FOV. In addition to position and orientation of the transducer, the lateral or azimuth extent and depth of the scanning define the FOV. Based on different settings, different sizes of FOV may be provided. The user or the system determines the FOV.

A two-dimensional image may be generated. B-mode frames of data are generated by B-mode scanning. A B-mode image represents the intensity or strength of return of acoustic echoes in the B-mode FOV. In other embodiments, other types of detection and corresponding scans are performed. For example, color flow (e.g., Doppler) estimation is used. Velocity, power, and/or variance are estimated. As another example, harmonic mode is used, such as imaging at a second harmonic of a fundamental transmit frequency. Combinations of modes may be used.

The initial scan or scans of the FOV are performed prior to separate scans of the ROI for quantitative imaging. The scanning is configured to cease scans of the FOV of the patient while scanning the ROI for quantification. Alternatively, B-mode imaging and quantitative imaging are interleaved.

The ROI is positioned in the FOV for QUS. The user, using a user interface, may position the FOV for the B-mode image and the ROI on the B-mode image. Alternatively, the ultrasound scanner, such as using an image processor or controller, determines a position of an ROI in the FOV of the ultrasound image.

In another implementation, AI guides the placement of the FOV and/or ROI in act 120. The AI is a machine-learned model. Any machine-learned model may be used, such as a fully connected neural network, a convolutional neural network, or a support vector machine. In one approach, a reinforcement machine-learned model is used to guide the user through a series of acts to position the FOV and/or ROI. In another approach, a neural network receives current images and detects landmarks or other indicators. The landmarks in the desired FOV and/or ROI are compared to the detected landmarks. The user adjusts the FOV and/or ROI until a match is found. In yet another approach, a neural network receives a series of images as the transducer is moved relative to the patient and places the FOV and/or ROI based on landmarks detected in the images or based on other image features.

The AI was previously machine trained. Training data is gathered, such as from patients that have undergone a same QUS imaging and/or longitudinal study. Using expert curation, objective measures, and/or other processes, ground truth data is created. The ground truth represents the FOV, landmarks, ROI, or other information reflecting a desired output of the AI. Given the sample inputs and corresponding ground truth outputs, the machine training optimizes values of learnable parameters in the defined model (e.g., neural network). Adam, gradient descent, or other optimization is performed so that the values of the learnable parameters of the model are set to generate outputs close to or agreeing with the ground truth given the range of input samples.

Once trained, the AI generates outputs in response to inputs. For example, a current ultrasound B-mode image is input. The AI outputs one or more landmarks, which may be compared to a table to identify whether the FOV is at the desired location relative to the patient. The AI may instead output an indication that the FOV is close, not close, or at the proper position. Alternatively, the AI may output instructions to the user to change the FOV, such as moving the transducer in a particular direction by a particular amount. Similar outputs may be used for placing the ROI.

Any input features, such as the ultrasound image, landmark locations, clutter levels by location, and/or fluid locations, may be used to place or guide placement of the FOV or ROI. The application of the machine-learnt model outputs landmark positions, user instructions, matches, and/or a position for the FOV and/or ROI. In an alternative, or additional, embodiment, the determination uses rules. For example, the ROI is positioned relative to but spaced away from a landmark while also avoiding clutter and fluid. The rules may indicate a specific orientation and distance from the landmark with tolerances for orientation and distance to account for avoiding clutter and fluid. Fuzzy logic may be used. The AI may identify the landmarks (e.g., fluid region, liver capsule, and liver segment) used by the rules to place the FOV and/or ROI.

The ROI may be positioned based on the type of QUS to be performed. For shear wave speed imaging of the liver, the ROI is positioned relative to a liver capsule. The ROI may be positioned based on the liver capsule and to avoid fluid and relatively higher clutter.

The orientation may be determined to include or avoid certain locations. The orientation may be based on the limits on steering from a transducer, detected landmarks that may cause acoustic shadowing, and/or directivity response of the tissue being quantified.

The ROI defining the scan region for quantitative imaging is less than the entire FOV of the B-mode image. The ROI is any size, such as 5 mm in lateral and 10 mm in axial. The ROI is sized to avoid fluid locations or relatively high clutter. Alternatively, the ROI is sized to include locations of relatively higher backscatter (e.g., lower clutter and lower noise).

The quantification scan may be affected by the size of the ROI. For shear wave imaging and other quantification scanning, the quantification relies on repetitive scanning of the ROI. By sizing the ROI smaller, the speed of scanning may increase, making the quantification less susceptible to motion artifact. By sizing the ROI larger, a more representative sampling for quantification may be provided. The ROI is sized as appropriate for the type of quantification. Different sizes may be selected based on a priority and avoidance of locations that may contribute to inaccuracy or artifacts.

The ROI is positioned for quantification of particular tissue or anatomy of interest. The size, shape, and orientation are set so that particular anatomy of the patient is within the ROI. Different anatomy or types of tissue may be included depending on the type of QUS imaging.

Once the FOV and ROI are placed, the ultrasound scanner performs the QUS in act 130. The ultrasound scanner (e.g., medical diagnostic ultrasound imaging system or scanner) performs QUS for a patient. The QUS may be limited to the ROI in the FOV of the scanner and/or transducer.

The ROI defines the locations of scanning for the quantitative imaging. For example, shear wave imaging is performed by the ultrasound scanner by scanning at the position of the ROI. Shear wave imaging may be used to quantify diagnostically useful information, such as the shear wave speed in tissue, Young's modulus, or a viscoelastic property. Shear wave imaging is a type of acoustic radiation force impulse (ARFI) imaging where ARFI is used to generate the shear wave, but other sources of stress and/or other types of ARFI (e.g., elasticity) imaging may be used.

Other types of quantitative imaging, such as strain, elasticity, backscatter, attenuation, and/or ultrasound-derived fat fraction, may be used. In one implementation, the QUS is ultrasound derived fat fraction. Various characteristics, such as attenuation and backscatter, are measured with ultrasound and used to derive an estimate of the fat fraction of liver tissue in the ROI.

The quantitative imaging results in a QUS image. The QUS image includes the values of the quantitative parameter or parameters for the ROI. For example, the shear wave velocity as a function of location in one, two, or three dimensions is included in the QUS image. In another example, the QUS image includes or is a quantitative value for the entire ROI.

The QUS image may include other information. For example, QUS values are used for the ROI and locations in the FOV outside the ROI are formed from the B-mode image. In one embodiment, the QUS image includes a reference volume, such as B-mode data in three dimensions as well as a two-dimensional B-mode image with overlaid QUS information for the ROI in the two-dimensional B-mode image.

The QUS image includes a graphic or defined ROI position. Alternatively, the locations of the QUS measurements indicate the position of the ROI. While a display of the QUS image may cover or not use B-mode data for locations within the ROI, the B-mode data being replaced may be provided as part of the QUS image even if not displayed.

In act 210, information usable for subsequent QUS imaging of the patient is stored. In one implementation, the QUS image with or without the ROI is stored. The QUS image with the ROI over a B-mode image provides the relative placement of the FOV and/or ROI. The ultrasound scanner, workstation, computer, or other processor stores the QUS image with the ROI in a memory, such as PACS memory, computerized medical record, or local memory (e.g., memory of the scanner).

In one implementation, the settings used to scan or for QUS imaging are stored. For example, the frequency, depth, type of transducer, gain, and/or other B-mode settings are stored. QUS settings may also or alternatively be stored. QUS settings may include the frequency, duration, power, amplitude, or other scan information for performing the desired measurements (e.g., attenuation and/or backscatter coefficient). For shear wave imaging, the frequency, duration, focal position, and/or other information for the pushing pulse may be stored. The values of settings for tracking (e.g., similar to B-mode values, including pulse repetition frequency) are stored.

Information for placing the FOV and/or ROI is stored. For example, the FOV may correspond to a particular or standard view. The identified view (view of a particular segment of the liver) is stored. As another example, the FOV and/or ROI relative to and/or including specific landmarks is stored. For example, the relative location of the gall bladder and/or liver capsule is stored. The QUS image with the ROI may be stored. The information usable by the AI to guide FOV and/or ROI placement is stored, such as a graphic of the ROI placed on a B-mode image, label of the view for the FOV, and/or relative location of landmarks to the FOV and/or ROI.

The information is stored in the memory of the ultrasound scanner. In another implementation, shown in FIG. 2, the information is stored at a remote storage 230, such as a storage separate from the memory of the ultrasound scanner. The remote storage may be in a different room, building, facility, city, or state than the ultrasound scanner. In one example, the storage is a PACS memory or a database for a computerized medical record. For the PACS memory, the information may be added to the header of one or more QUS images stored for the patient. For example, the scan settings, FOV, and/or ROI information are stored in the header of a Digital Imaging and Communication in Medicine (DICOM) QUS image or images. As another example, the information is stored as metadata with a report, QUS image, patient medical record, or other medical information. Dedicated storage may be used for the information in other implementations.

In act 220, the ultrasound scanner scans a patient in a subsequent examination. This follow-up examination is a separate appointment, such as being on a different day than the previous or baseline scan of act 200. This subsequent examination in act 220 may be after treatment, while the baseline examination in act 200 may be before treatment. While the scans of acts 200 and 220 may be on a same day, an event occurs between them separating the two examinations in time and workflow.

The scan of act 220 in FIG. 2 corresponds to the method shown in FIG. 1. The same or different ultrasound scanner is used for acts 200 and 220. The same type of ultrasound scanner is used. The settings used for the baseline or previous scan of act 200 may be used by the ultrasound scanner for the follow-up or subsequent scan of act 220.

In act 100, a processor (controller) of the ultrasound scanner acquires values for settings used in the previous QUS examination of the patient. The settings used in the baseline QUS scan of act 200 are accessed from memory, such as the remote storage 230. The values are acquired from local memory or a memory external to the ultrasound scanner, such as a PACS database. The remote storage 230 may be cloud storage, such as a database accessible through a computer network (e.g., the Internet, local area network, or wide area network).

The settings are acquired in response to indication of the patient to be scanned and/or entry of an order for scanning the patient. In alternative embodiments, the user (sonographer) acquires the settings by selection from a menu.

In act 102, the settings for the ultrasound scanner from the previous scan session are reloaded. The ultrasound scanner places the values for configurable settings of the scanner in a buffer for reloading. System settings are reloaded from one or more previous scan sessions.

Using the same values for the settings facilitates longitudinal ultrasound scanning: scanner settings are consistent from baseline to follow-up. Consistent scanning parameters and settings are maintained across sessions by acquiring the stored values for settings from the previous (e.g., baseline) scan. Since the values of the settings may impact the sample points, FOV, ROI, and echoes used for measurement, using the same values of settings ensures alignment and comparison of scans over different time points.

In one implementation, the values of the settings are acquired from metadata of a stored QUS image from the previous QUS examination of the patient. The metadata may be formatted so that particular locations contain the values of the settings. Alternatively, the metadata is searched to find the values of the settings. The QUS image from the previous examination is accessed from the memory, such as the remote storage 230. The values are then acquired from the metadata (e.g., header) of the image. In other implementations, the values of the settings are stored separately from the QUS image, such as part of the medical record or radiology report for the patient. The settings are acquired from the medical record or radiology report. The settings may be detected or mined from the QUS image, such as where the image is annotated with or includes the settings.

Values of any of various settings may be acquired. For example, the (1) frequency, gain, and/or depth for B-mode imaging as a background for the previous QUS examination, (2) frequency, gain, and/or depth for tracking for QUS examination, and/or (3) push-pulse frequency, amplitude, and/or duration for the previous QUS examination are acquired. Values for settings of other scan parameters, such as scan format, focal depth, sample locations, sample frequency, and/or pulse magnitude may be stored in act 210 and acquired in act 100. Filtering or back-end processing settings, such as spatial and/or temporal filtering, may be stored in act 210 and acquired in act 100. In one implementation, values from the previous scan of act 200 are acquired for all the scan and/or filtering settings.

Different types of transducers may be connected to the ultrasound scanner. The type of transducer used in the baseline or previous QUS scan of act 200 may be stored in act 210 and acquired in act 100. The processor compares the type of the currently connected transducer to the type used in the previous QUS examination. The same type of transducer is to be used. A notice to the user may be generated to indicate the type of transducer to connect to the ultrasound scanner for the follow-up scan of act 220.

In act 110, the processor configures the ultrasound scanner for the current (e.g., follow-up) QUS examination of the patient. The processor configures the ultrasound scanner to scan the same in the follow-up examination of act 220 as was done in the previous examination of act 200. In an alternative, the user enters or selects the values for one or more settings based on the previous values.

The values for the settings as acquired are used. The processor sets the values of the settings for the scan of act 220 to the acquired values. The reloaded values from the previous QUS examination are used. The values of the settings to be used in the current QUS examination are set to the values from the previous QUS examination. The buffer or other memory accessed by the ultrasound scanner during scanning to control the scan of act 220 is loaded with the values from the previous QUS.

For the transducer, the processor verifies that the same type of transducer is connected. If a different type of transducer is being used or currently connected, then the user is notified to change the type of transducer.

In act 120, the processor guides placement of a ROI relative to the patient for the current QUS scan of act 220. The same guidance used in the previous or baseline scan of act 200 is applied in the follow-up or subsequent scan of act 220. By using the same guidance, the ROI is positioned to measure the same tissue. The operator is guided to the optimal scanning positions to minimize variability between different users and sessions, improving measurement accuracy and reducing variability by providing real-time guidance and/or automated adjustments of measurement ROI for optimal acquisitions of QUS measurements (pSWE, UDFF, etc.).

Placement from the previous use may be used to guide the current placement. The FOV and/or ROI placement information is acquired with the values of the settings in act 100. For example, the placement is provided as the QUS image showing the ROI and surrounding tissue or as placement relative to landmarks and/or a known view. The placement is acquired from the remote (e.g., cloud) storage 230 or local memory.

The information from the previous placement of the FOV and/or ROI may be used to guide in the follow-up scan of act 220. The machine-learned model or other AI guides using the same criteria with additional inputs for previous FOV and/or ROI placement. Different or the same AI or machine-learned models may be used, such as using a different AI where additional information is available (e.g., previously used placement information). The processor applies the AI to guide a current position of the ROI relative to a patient based on data from the previous scan session. For example, the previous ROI was placed at a particular liver segment at a position and/or orientation relative to the liver capsule in a given view. The AI uses inputs of the current images and the past placement information to guide placement of the current ROI over the same liver segment at the position and/or orientation relative to the liver capsule where the FOV is placed to be at the same view.

The AI guides a user to the optimal position leveraging data from previous scan session. The AI may be used to identify anatomical structures, to guide the user to the optimal view and/or to place the ROI matching the baseline QUS examination.

In one implementation, the machine-learned model is used to detect specific landmarks for matching. The user repositions the FOV and/or ROI based on feedback derived from the AI-detected landmarks so that the detected landmarks match the landmarks of the previous QUS examination.

In another implementation, the ROI for QUS is automatically positioned by a processor of the ultrasound scanner. As the user performs surveillance scanning (e.g., B-mode scans while moving the transducer to find the FOV), the ROI is automatically positioned on each image. The indicator shows the degree of similarity (e.g., as a percentage, colored bar, color highlight, or other indication) between the current view (i.e., current FOV or B-mode image) and the reference view (i.e., previous FOV or B-mode image). In another approach, each current image is searched for different ROI positions to find the ROI position in the current FOV with a greatest or sufficient similarity to the ROI of the previous view. As different FOVs occur during searching, the indicator is the degree of similarity for the best ROI of the current image to the ROI of the image from the previous QUS examination. The user uses the indication of similarity to position the FOV and/or ROI in the current examination.

The similarity is measured between B-mode or other modes of data and/or landmark positions. The ROI and/or FOV with a greatest similarity is based on comparison of the B-mode, landmark positions, and/or other ultrasound data from one FOV and/or ROI to another FOV and/or ROI.

Any similarity measure may be used. For example, auto-correlation is used. As another example, a minimum sum of absolute differences is used. In another example, auto-correlation in conjunction with algorithms for recognizing anatomical structures is used. For example, the organ is identified. The indicator is weighted based on the organ. Where the ROI is for the liver, the organ identified as other than the liver in a FOV may provide indication of no match. Where the organ is identified as the liver, then an organ match is indicated. Further refining is provided using similarity of B-mode data and/or relative locations of landmarks.

In another implementation, the machine-learned model outputs an indicator of similarity. The model was trained to indicate level or degree of match. The past QUS image showing the ROI and the current image and ROI placement are input to the model, which outputs an indication of the current ROI matching the past ROI. The user or the processor maximizes this indication by repositioning the current ROI and/or FOV.

In another implementation, the machine-learned model detects one or more landmarks for a current position of the ROI. The current image and ROI are input to the model. The responsive output is detected landmarks and their position relative to the current ROI. The processor compares the landmarks relative to the current ROI to the landmarks relative to the past ROI. Where the relative landmark position to the current ROI matches the relative landmark position to the past ROI, the same ROI position is identified. The current ROI covers the same tissue in the patient.

In yet another implementation, the current B-mode image and ROI placement is input to the machine-learned model. The machine-learned model includes a memory of previous inputs and outputs instructions for moving the FOV and/or ROI. This machine-learned model was trained as a reinforcement learning model to output acts or instructions for altering ROI and/or FOV to lead to the same FOV and/or ROI placement in the current imaging as for the past QUS examination.

Once the FOV and/or ROI are placed using the guidance of act 120 and the scanner is configured in act 110, the QUS examination is performed in act 130. The current QUS examination is performed in the same way as the previous QUS examination. For example, the baseline scan of act 200 was for shear wave velocity and/or ultrasound-derived fat fraction quantification for the liver of the patient. The current or follow-up scan of act 220 is also for shear wave velocity and/or ultrasound-derived fat fraction quantification for the same part of the liver of the patient. By using the same settings, FOV, and ROI, the measurements from different times may be compared. Less human variability exists even where different sonographers are used, so the measurements more accurately reflect the same tissue characteristic.

The ultrasound scanner performs this follow-up QUS examination. The FOV and/or ROI are located automatically, semi-automatically, or manually. The current ROI is placed and used for QUS measurements to measure at the same location as the previous ROI using the same scanner settings. Since the current ROI is positioned on at least some of the same anatomy as the past ROI, the QUS measurements are for at least some of the same anatomy.

A QUS image is generated. The QUS image shows values for a QUS parameter. For example, shear wave velocity, attenuation, or backscatter is determined from ultrasound data. The same type of QUS examination is performed for the current examination as for a past examination. Since the ROI is for the same anatomy, at least in part, then the values of the QUS parameter or parameters may reflect changes in the anatomy due to treatment.

In act 140, the processor displays a comparison of one or more measurements from the current QUS examination with the same one or more measurements from the previous QUS examination. The comparison may be the measurement from different times displayed at a same time. For example, a graph, chart, or representation of the values of the measurements over time or pre and post treatment are displayed. In other implementations, the comparison is a difference or change. The change is calculated from the measurements at different times, and the change is displayed.

The comparison may be displayed with, an annotation on, or over a QUS image. The image is displayed on a display device. The image processor, renderer, or other device generates an image from the QUS imaging for the ROI. The image includes one or more quantities representing tissue characteristics. An alphanumeric or graphical representation of one or more quantities may be provided, such as a shear wave speed Vₛ for the ROI overlaid as an annotation with a B-mode image. Alternatively, or additionally, the quantities for different locations are displayed. For example, the quantities for different locations in the ROI modulate the brightness and/or color so that spatial representation of the quantity is provided in the image. The spatial representation may be overlaid or included in a B-mode or other image. The quantity or quantities may be provided without other types of imaging or may be added to or overlaid with other types of ultrasound imaging.

QUS images and corresponding measurements from different times may be displayed together or at a same time. This comparison is provided by both the QUS images and measurements from different times.

In one implementation, the display is on a display of the ultrasound scanner. A quantitative analysis for the QUS imaging by the ultrasound scanner using the reloaded or same settings with a same position of the ROI is performed.

In another implementation, an analysis application or tool 240 generates the display. The analysis application or tool 240 may be executed by the processor of the ultrasound scanner or a device remote from the ultrasound scanner. For example, a mobile phone, tablet, laptop computer, desktop computer, workstation, or another processor separate from the ultrasound scanner runs the application or tool 240. Using access to the QUS images, values of settings, and/or FOV/ROI placement information in the remote storage 230, the application or tool 240 may be operated on various devices.

The application or tool 240 is a common quantitative analysis and reporting program across platforms. Analysis and visualization of changes in QUS measurements may be displayed. Longitudinal data is analyzed to track changes over time. A user interface accessible through system software, mobile application, or web application allows display of longitudinal study information for the patient. The application or tool 240 ensures secure data sharing and access between patients and healthcare providers for comprehensive care management. The application or tool 240 may be used by physicians, sonographers, and/or patients, such as providing guidance to clinical users on interpretations of UDFF and pSWE values (e.g., recommendation 241).

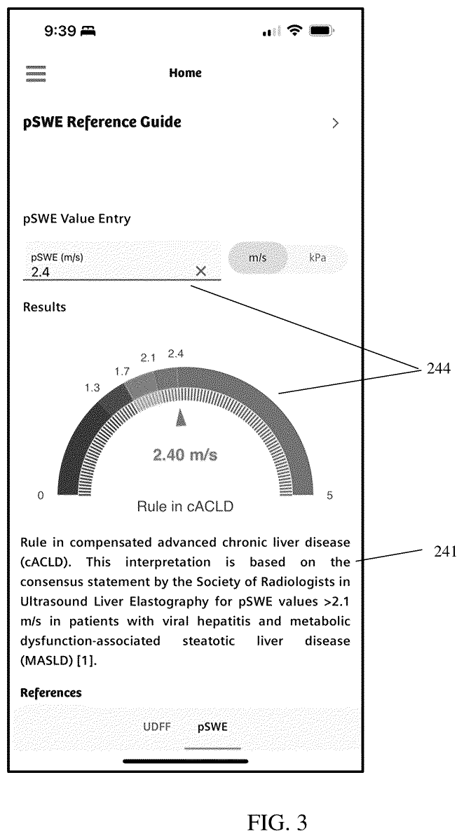

FIG. 3 shows an example screen of a mobile device executing the application or tool 240. A value of the shear wave speed (pSWE) for a current QUS examination of a patient is shown at 244 as an alphanumeric value and as a chart or graph. The chart or graph provides the location of the current value of the speed measurement relative to a range of possible values. Where values from measurements at different times are displayed, both values are shown but color coded or labeled to indicate the different times. Alternatively, or additionally, a change is displayed.

The application or tool 240 may provide other information. For example, a recommendation 241 is displayed. The recommendation may be an explanation of the measurement or comparison. FIG. 3 shows an example explaining the range of values and diagnostic information for the measured value within that range.

As another example, effectiveness 242 is displayed. The effectiveness may be an amount of change after treatment. Other information, such as expected change, may be displayed.

In another example, the effects of diet and/or exercise 243 is displayed. The change in the value of the measurement over time while the patient is following a diet and/or exercise routine monitors the effectiveness of the diet and/or exercise. Users may monitor progress by visualizing the progress.

The application or tool 240 may generate notices 245 to the user. For example, notifications for scheduled scans and recommended scanning locations are generated.

A report 244 may be used for future scan sessions. The report 244 may store and process historical scan data to inform and improve the accuracy of future scan sessions. The values of settings and/or information for placement of the FOV and/or ROI may be stored. The values of measurements may be stored.

The QUS ultrasound imaging is used for diagnosis, prognosis and/or treatment guidance. Enhanced, more consistent, and/or more accurate quantitative imaging due to proper ROI placement for different examinations leads to better diagnosis, prognosis, and/or treatment by a physician. The physician and patient benefit from the improvement as the output of the quantification is more likely reflective of the same anatomy.

FIG. 4 shows one embodiment of a system 400 for longitudinal monitoring in QUS imaging. The system 400 is used for an initial or earlier QUS examination and/or for a subsequent or later QUS examination. The system 400 provides for storage of values of settings, FOV placement, and/or ROI placement. The system 400 provides for reloading of the values and use of the placement to guide subsequent FOV and ROI placement. The QUS imaging is more repeatable with less human-based variation by using the same settings and guided ROI and/or FOV placement.

The system 400 is an ultrasound imager or scanner. In one embodiment, the ultrasound scanner is a medical diagnostic ultrasound imaging system. In alternative embodiments, the ultrasound imager is a personal computer, workstation, PACS station, or another arrangement at a same location or distributed over a network for real-time or post-acquisition imaging.

The system 400 implements the method of FIG. 1, the method of FIG. 2, or other methods. The system 400 includes a transmit beamformer 410, a transducer 420, a receive beamformer 430, an image processor 440, a display 450, and a memory 460. Additional, different, or fewer components may be provided. For example, a spatial filter, a scan converter, a mapping processor for setting dynamic range, and/or an amplifier for application of gain are provided. As another example, a user input is provided.

The transmit beamformer 410 is configured to generate waveforms for a plurality of channels with different or relative amplitudes, delays, and/or phasing to focus a resulting beam at one or more depths. The waveforms are generated and applied to elements of the array forming the transducer 420 with any timing or pulse repetition frequency. The transmit beamformer 410 is configured to generate waveforms for B-mode scanning and QUS scanning (e.g., pushing pulse and tracking pulses).

Upon transmission of acoustic waves from the transducer 420 in response to the generated waves, one or more beams are formed during a given transmit event. The beams are for B-mode, quantitative mode (e.g., ARFI or shear wave imaging), or another mode of imaging. Sector, Vector®, linear, or other scan formats may be used. The same region is scanned multiple times for generating a sequence of images or for quantification.

The transducer 420 is a 1-, 1.25-, 1.5-, 1.75- or 2-dimensional array of piezoelectric or capacitive membrane elements. The transducer 420 includes a plurality of elements for transducing between acoustic and electrical energies.

The transducer 420 is releasably connectable with the transmit beamformer 410 for converting electrical waveforms into acoustic waveforms, and with the receive beamformer 430 for converting acoustic echoes into electrical signals. The transducer 420 transmits the transmit beams where the waveforms have a frequency and are focused at a tissue region or location of interest in the patient. Receive signals are generated in response to ultrasound energy (echoes) impinging on the elements of the transducer 420.

The receive beamformer 430 applies relative delays, phases, and/or apodization to form one or more receive beams in response to each transmission for detection. The receive beamformer 430 outputs data representing spatial locations using the received acoustic signals.

For ARFI or shear wave imaging, parallel receive beamformation is used in tracking. For tracking displacements, a transmit beam covering the ROI is transmitted. Two or more (e.g., 8, 16, 32, or 64) receive beams distributed evenly or unevenly in the ROI are formed in response to each transmit beam.

The image processor 440 detects, such as detecting intensity, from the beamformed samples. Any detection may be used, such as B-mode and/or color flow detection. In one embodiment, a B-mode detector is a general processor, application specific integrated circuit, or field programmable gate array. Log compression may be provided by the B-mode detector so that the dynamic range of the B-mode data corresponds to the dynamic range of the display. The image processor 440 may or may not include a scan converter.

The image processor 440 quantifies, such as determining shear wave speed based on ultrasound measurement of tissue displacement due to a shear wave. Other quantification from the receive signals to determine a tissue characteristic may be performed, such as determining attenuation, backscatter, and/or ultrasound-derived fat fraction.

For longitudinal study of a patient, the ultrasound imaging system 400 is configured to store settings, FOV placement, and/or ROI placement information in the memory 460 and to reload the settings and access FOV placement and ROI placement from the memory 460. For example, the settings (values of settings) from a baseline QUS imaging session for a patient are reloaded for a follow-up QUS imaging session for the patient. The reload is triggered by user selection of a reload option. Alternatively, the reload occurs automatically in response to identification of the patient and/or selection of a QUS imaging order for the patient.

The image processor 440 includes a controller, general processor, application specific integrated circuit, field programmable gate array, graphics processing unit, tensor processor, artificial intelligence processor, or another processor to guide positioning of the FOV and/or ROI for QUS imaging. The image processor 440 includes or interacts with a beamformer controller to scan the ROI in the QUS scanning. The image processor 440 is configured by hardware, software, and/or firmware.

The image processor 440 may be configured by a machine-learned model to identify an anatomical structure and/or guide a user to a view of the patient and position of the ROI in the view. The machine-learned model is configured by previous training to output (1) the locations of landmarks, (2) similarity between FOVs and/or ROIs, (3) instructions to change FOV and/or ROI, or (4) another output to guide the placement of the ROI and/or FOV. The image processor 440 may guide the user or itself to place or automatically place the ROI using the output of the machine-learned model. The view and position for the follow-up QUS imaging session is guided to match the baseline QUS imaging session. The information about past placement is used by the machine-learned model and/or the image processor to guide placement for the current QUS imaging.

The display 450 is a CRT, LCD, monitor, plasma, projector, printer, or other device for displaying an image or sequence of images. Any now known or later developed display 450 may be used. The display 450 displays a B-mode image, a QUS image (e.g., annotation or color modulation on a B-mode image), analysis application or tool user interface, or other QUS information. The display 450 displays one or more images representing the ROI or tissue characteristic in the ROI.

The display 450 is configured by loading an image into a display plane or buffer. In one implementation, the display 450 is configured to display a change in a QUS measurement from the baseline QUS imaging session to the follow-up QUS imaging session. Other comparisons may be displayed. A history of measurements over multiple (e.g., three or more) examinations or time may be displayed.

In one implementation, the display 450 is remote or separate from any display of the ultrasound scanner. For example, the display 450 is a display of a mobile device or computer. The user interface of a mobile or web application or tool is displayed with the comparison.

The memory 460 is a local memory or a remote memory. As a remote memory, the memory 460 connects to the ultrasound imaging system through a computer network. The memory 460 is configured to store the settings, view (FOV), and ROI position for the baseline and/or another QUS imaging session. The memory 460 is accessed for reloading settings, guiding FOV and/or ROI placement, and/or display of comparison (longitudinal QUS measurements).

The image processor 440, and/or the ultrasound system 400 operate pursuant to instructions stored in the memory 460 or another memory. The instructions configure the system for performance of the acts of FIG. 1 or FIG. 2. The instructions configure for operation by being loaded into a controller, by causing loading of a table of values (e.g., elasticity imaging sequence), and/or by being executed. The memory is a non-transitory computer readable storage media. The instructions for implementing the processes, methods and/or techniques discussed herein are provided on the computer-readable storage media or memories, such as a cache, buffer, RAM, removable media, hard drive, or other computer readable storage media. Computer readable storage media include various types of volatile and nonvolatile storage media. The functions, acts, or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

Listed below are various Illustrative Embodiments. The Illustrative Embodiments summarize different combinations of aspects or features. Other combinations of any of the aspects or features with any other one or more of the aspects or features may be provided. Aspects or features from one type (e.g., method or system) may be used in another type (system or method).

Illustrative Embodiment 1. A method for longitudinal monitoring in quantitative ultrasound imaging with an ultrasound scanner, the method comprising: acquiring first values for settings used in a previous quantitative ultrasound examination of a patient; configuring the ultrasound scanner for a current quantitative ultrasound examination of the patient with the first values for the settings; guiding, by a machine-learned model, placement of a region of interest relative to the patient; performing the current quantitative ultrasound examination of the patient with the ultrasound scanner as configured by the first values and using the region of interest as placed by the guidance; and displaying a comparison of a first measure from the current quantitative ultrasound examination with a second measure from the previous quantitative ultrasound examination.

Illustrative Embodiment 2. The method of Illustrative Embodiment 1, wherein acquiring comprises loading the first values from meta data of a stored ultrasound image from the previous quantitative ultrasound examination of the patient.

Illustrative Embodiment 3. The method of any of Illustrative Embodiments 1-2, wherein acquiring comprises acquiring by a processor of the ultrasound scanner, and wherein configuring comprises configuring by the processor.

Illustrative Embodiment 4. The method of any of Illustrative Embodiments 1-3, wherein acquiring comprises acquiring the first values for the settings where the settings comprise frequency, gain, and/or depth for B-mode imaging as a background for the previous quantitative ultrasound examination and push-pulse frequency and/or duration for the previous quantitative ultrasound examination.

Illustrative Embodiment 5. The method of any of Illustrative Embodiments 1-4, wherein acquiring comprises acquiring a type of transducer used in the previous quantitative ultrasound examination, and wherein configuring comprises verifying connection of the type of transducer to the ultrasound scanner for the current quantitative ultrasound examination.

Illustrative Embodiment 6. The method of any of Illustrative Embodiments 1-5, wherein configuring comprises reloading the first values from the previous quantitative ultrasound examination into the settings for the ultrasound scanner.

Illustrative Embodiment 7. The method of any of Illustrative Embodiments 1-6, wherein guiding comprises displaying an indicator of a current position of the region of interest matching a previous position of the region of interest used in the previous quantitative ultrasound examination, the indicator output by the machine-learned model.

Illustrative Embodiment 8. The method of any of Illustrative Embodiments 1-7, wherein guiding comprises detecting one or more current landmarks for a current position of the region of interest by the machine-learned model, and indicating whether the current landmarks match previous landmarks for a previous position of the region of interest from the previous quantitative ultrasound examination.

Illustrative Embodiment 9. The method of any of Illustrative Embodiments 1-8, wherein guiding comprises displaying instructions for a user to move a current position of the region of interest, the machine-learned model comprising a machine-learned reinforcement learning model configured by training to output the instructions.

Illustrative Embodiment 10. The method of any of Illustrative Embodiments 1-9, wherein performing comprises performing the current quantitative ultrasound examination as a shear wave velocity and/or ultrasound-derived fat quantification for a liver of the patient.

Illustrative Embodiment 11. The method of Illustrative Embodiment 10, wherein guiding comprises guiding the placement to be at a same liver segment and position relative to a liver capsule of the patient for the current quantitative ultrasound examination as the previous quantitative ultrasound examination.

Illustrative Embodiment 12. The method of any of Illustrative Embodiments 1-11, wherein displaying comprises displaying a change from the second measure to the first measure.

Illustrative Embodiment 13. The method of any of Illustrative Embodiments 1-12, wherein the first values and previous position of the region of interest relative to the patient of the previous quantitative ultrasound examination are stored externally to the ultrasound scanner, wherein displaying comprises displaying on the ultrasound scanner, a mobile application, or a web application.

Illustrative Embodiment 14. The method of any of Illustrative Embodiments 1-13, wherein displaying the comparison further comprises displaying an explanation of the first measure and/or comparison.

Illustrative Embodiment 15. A method for longitudinal monitoring in quantitative ultrasound imaging with an ultrasound scanner, the method comprising: reloading settings for the ultrasound scanner from a previous scan session; guiding, by an artificial intelligence, a current position of a region of interest relative to a patient based on data from the previous scan session; and displaying quantitative analysis from the quantitative ultrasound imaging by the ultrasound scanner using the reloaded settings with the current position of the region of interest.

Illustrative Embodiment 16. The method of Illustrative Embodiment 15, wherein reloading comprises reloading from cloud storage, wherein guiding comprises guiding where the data from the previous scan session is from the cloud storage, and wherein displaying comprises displaying on the ultrasound scanner, a mobile device, or a computer where the quantitative analysis is from the cloud storage.

Illustrative Embodiment 17. A system for longitudinal monitoring in quantitative ultrasound imaging, the system comprising: an ultrasound imaging system configured to reload settings from a baseline quantitative ultrasound imaging session for a follow-up quantitative ultrasound imaging session; an image processor configured by a machine-learned model to identify an anatomical structure and guide a user to a view of the patient and position of a region of interest in the view, the view and position for the follow-up quantitative ultrasound imaging session to match the baseline quantitative ultrasound imaging session; and a display configured to display a change in a quantitative ultrasound measurement from the baseline quantitative ultrasound imaging session to the follow-up quantitative ultrasound imaging session.

Illustrative Embodiment 18. The system of Illustrative Embodiment 17, wherein the ultrasound imaging system is configured to reload in response to identification of the patient and/or selection of a quantitative ultrasound imaging order for the patient.

Illustrative Embodiment 19. The system of any of Illustrative Embodiments 17-18, further comprising a memory communicatively connected to the ultrasound imaging system through a computer network, the memory configured to store the settings, view, and position for the baseline quantitative ultrasound imaging session.

Illustrative Embodiment 20. The system of any of Illustrative Embodiments 17-19, wherein the display comprises a display of a mobile application or web application separate from the ultrasound imaging system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for longitudinal monitoring in quantitative ultrasound imaging with an ultrasound scanner, the method comprising:
acquiring first values for settings used in a previous quantitative ultrasound examination of a patient;
configuring the ultrasound scanner for a current quantitative ultrasound examination of the patient with the first values for the settings;
guiding, by a machine-learned model, placement of a region of interest relative to the patient;
performing the current quantitative ultrasound examination of the patient with the ultrasound scanner as configured by the first values and using the region of interest as placed by the guidance; and
displaying a comparison of a first measure from the current quantitative ultrasound examination with a second measure from the previous quantitative ultrasound examination.

2. The method of claim 1, wherein acquiring comprises loading the first values from meta data of a stored ultrasound image from the previous quantitative ultrasound examination of the patient.

3. The method of claim 1, wherein acquiring comprises acquiring by a processor of the ultrasound scanner, and wherein configuring comprises configuring by the processor.

4. The method of claim 1, wherein acquiring comprises acquiring the first values for the settings where the settings comprise frequency, gain, and/or depth for B-mode imaging as a background for the previous quantitative ultrasound examination and push-pulse frequency and/or duration for the previous quantitative ultrasound examination.

5. The method of claim 1, wherein acquiring comprises acquiring a type of transducer used in the previous quantitative ultrasound examination, and wherein configuring comprises verifying connection of the type of transducer to the ultrasound scanner for the current quantitative ultrasound examination.

6. The method of claim 1, wherein configuring comprises reloading the first values from the previous quantitative ultrasound examination into the settings for the ultrasound scanner.

7. The method of claim 1, wherein guiding comprises displaying an indicator of a current position of the region of interest matching a previous position of the region of interest used in the previous quantitative ultrasound examination, the indicator output by the machine-learned model.

8. The method of claim 1, wherein guiding comprises detecting one or more current landmarks for a current position of the region of interest by the machine-learned model, and indicating whether the current landmarks match previous landmarks for a previous position of the region of interest from the previous quantitative ultrasound examination.

9. The method of claim 1, wherein guiding comprises displaying instructions for a user to move a current position of the region of interest, the machine-learned model comprising a machine-learned reinforcement learning model configured by training to output the instructions.

10. The method of claim 1, wherein performing comprises performing the current quantitative ultrasound examination as a shear wave velocity and/or ultrasound-derived fat quantification for a liver of the patient.

11. The method of claim 10, wherein guiding comprises guiding the placement to be at a same liver segment and position relative to a liver capsule of the patient for the current quantitative ultrasound examination as the previous quantitative ultrasound examination.

12. The method of claim 1, wherein displaying comprises displaying a change from the second measure to the first measure.

13. The method of claim 1, wherein the first values and previous position of the region of interest relative to the patient of the previous quantitative ultrasound examination are stored externally to the ultrasound scanner, wherein displaying comprises displaying on the ultrasound scanner, a mobile application, or a web application.

14. The method of claim 1, wherein displaying the comparison further comprises displaying an explanation of the first measure and/or comparison.

15. A method for longitudinal monitoring in quantitative ultrasound imaging with an ultrasound scanner, the method comprising:
reloading settings for the ultrasound scanner from a previous scan session;
guiding, by an artificial intelligence, a current position of a region of interest relative to a patient based on data from the previous scan session; and
displaying quantitative analysis from the quantitative ultrasound imaging by the ultrasound scanner using the reloaded settings with the current position of the region of interest.

16. The method of claim 15, wherein reloading comprises reloading from cloud storage, wherein guiding comprises guiding where the data from the previous scan session is from the cloud storage, and wherein displaying comprises displaying on the ultrasound scanner, a mobile device, or a computer where the quantitative analysis is from the cloud storage.

17. A system for longitudinal monitoring in quantitative ultrasound imaging, the system comprising:
an ultrasound imaging system configured to reload settings from a baseline quantitative ultrasound imaging session for a follow-up quantitative ultrasound imaging session;
an image processor configured by a machine-learned model to identify an anatomical structure and guide a user to a view of the patient and position of a region of interest in the view, the view and position for the follow-up quantitative ultrasound imaging session to match the baseline quantitative ultrasound imaging session; and
a display configured to display a change in a quantitative ultrasound measurement from the baseline quantitative ultrasound imaging session to the follow-up quantitative ultrasound imaging session.

18. The system of claim 17, wherein the ultrasound imaging system is configured to reload in response to identification of the patient and/or selection of a quantitative ultrasound imaging order for the patient.

19. The system of claim 17, further comprising a memory communicatively connected to the ultrasound imaging system through a computer network, the memory configured to store the settings, view, and position for the baseline quantitative ultrasound imaging session.

20. The system of claim 17, wherein the display comprises a display of a mobile application or web application separate from the ultrasound imaging system.
